(19) United States
(12) Patent Application Publication
(10) Pub. No.: US 2011/0098667 A1
(43) Pub. Date: Apr. 28, 2011

(54) INCONTINENCE GARMENT

(75) Inventors: Joseph J. Nicholson, New York, NY (US); Nancy Weber, New York, NY (US)

(73) Assignee: Dollface Designs, LLC

(21) Appl. No.: 12/910,737

(22) Filed: Oct. 22, 2010

Related U.S. Application Data
(60) Provisional application No. 61/254,524, filed on Oct. 23, 2009.

(51) Int. Cl. A61F 13/496

(52) U.S. Cl. 604/385.3

(57) ABSTRACT

An incontinence garment comprising: a waistband; a front portion attached to the waistband, wherein the front portion has at least a first securing member disposed thereon; a back portion attached to the waistband in a position opposite to the front portion, wherein the back portion has at least a second securing member disposed thereon; a crotch portion disposed between the front portion and the back portion and opposite to the waistband, wherein the first and second securing members are disposed between the waistband and the crotch portion; and an absorbent assembly disposed between the front portion and second back portion, wherein the absorbent assembly comprises a first end affixed to the first securing member and a second end affixed to the second securing member.

### CROSS-REFERENCED APPLICATIONS

The present application claims priority to U.S. Provisional Application No. 61/254,524, filed on Oct. 23, 2009, which is incorporated herein in its entirety by reference thereto.

### BACKGROUND

1. Field of the Disclosure

The present disclosure relates generally to an incontinence garment. More particularly, the present disclosure relates to an incontinence garment that uniquely holds and conceals an absorbent pad or other moisture collecting device.

2. Discussion of the Background Art

Urinary incontinence (UI) is the loss of bladder control or the leakage of urine. UI is a common problem amongst men and woman worldwide, with about 25 million adults in the U.S. alone suffering from some degree of symptoms (about 5 million of those are men). This disease can occur at any age, but is more prevalent amongst the elderly. In addition, UI can be a temporary or short-term problem resulting from surgery or health condition. Symptoms can range from mild leakage to uncontrollable and embarrassing wetting. This often has a profound affect on one's quality of life and their social, sexual, interpersonal and professional capacity.

Traditionally, those suffering from UI wear an absorbent product to manage symptoms and protect clothing. The products currently available can often be bulky, costly, time consuming and for many people embarrassing. This includes inserts, pads, adult diapers, plastic-coated underwear and belted undergarments. Adult diapers are frequently selected since they have a high absorbency, come in a number of sizes and are available in either disposable or reusable form. However, adult diapers can have an uncomfortable design containing plastic wings, tapes, elastic leg straps or gathers to remain in place. In addition, they often fit snugly against the body causing the wearer irritation.

Recently, products have been introduced that resemble normal underwear for both men and women. These are available to provide more discreet protection and are often re-usable, washable and more comfortable for the wearer. However, the current products still contain many limitations and undesirable features. This includes the use of a plastic material to compose the product or to form a sewn in pouch to accommodate a pad or insert. As a result, this still gives the wearer the sense of wearing a diaper.

Furthermore, many products require the purchase of a pad or insert which was specifically designed and manufactured for that particular undergarment. This can be both expensive and inconvenient for the wearer.

Therefore, a need exists for a reusable stylish garment, which is indistinguishable from a traditional undergarment, but which effectively holds and conceals nearly any brand of absorbent pad.

Additional objects, advantages and novel features of the present disclosure will be set forth in part in the description which follows, and in part will become apparent to those skilled in the art upon examination of the following or may be learned by practice of the present disclosure. The objects and advantages of the present disclosure may be realized and attained by means of the instrumentalities and combinations particularly pointed out in the appended claims.

### SUMMARY

An incontinence garment comprising: a waistband; a front portion attached to the waistband, wherein the front portion has at least a first securing member disposed thereon; a back portion attached to the waistband in a position opposite to the front portion, wherein the back portion has at least a second securing member disposed thereon; a crotch portion disposed between the front portion and the back portion and opposite to the waistband, wherein the first and second securing members are disposed between the waistband and the crotch portion; and an absorbent assembly disposed between the front portion and second back portion, wherein the absorbent assembly comprises a first end affixed to the first securing member and a second end affixed to the second securing member.

The first and second securing members are formed of an elastic type material. The first end of the absorbent assembly is affixed to the first securing member via a first fastener, and the second end of the absorbent assembly is affixed to the second securing member via a second fastener. The first and second fasteners are at least one fastener selected from the group consisting of: hook and loop type fasteners, adhesives, snaps, and buttons.

The first and second fasteners are hook and loop type fasteners, wherein each the first and second securing members have respective hook portions from the hook and loop type fastener affixed thereto and each the first and second ends of the absorbent assembly have respective loop portions affixed thereto. Preferably, the absorbent assembly includes an absorbent pad. Preferably, the hook portions are affixed to a surface of the first and second securing members, respectively, wherein the surface is that surface which faces toward the user's body.

### BRIEF DESCRIPTION OF THE DRAWINGS

Other and further benefits, advantages and features of the present invention will be understood by reference to the following specification in conjunction with the accompanying drawings, in which like reference characters denote like elements of structure and:

FIG. 1 illustrates a front view of an incontinence garment according to the present disclosure.

FIG. 2 illustrates a plan view of the interior of the incontinence garment with a first band and a second band to secure an absorbent pad.

FIG. 3 illustrates a view of a fastening component attached to the interior of a second band of the incontinence garment.

FIG. 4 illustrates a plan view of the interior of the incontinence garment having a fastening component attached to the interior of a first band and a second band with an absorbent pad secured within.

FIG. 5 illustrates a plan view of the interior of the incontinence garment having a fastening component attached to the exterior of a first band and a second band with an absorbent pad secured within.

FIG. 6 illustrates a view of a fastening component attached to the exterior of a first band of the incontinence garment.

FIG. 7 illustrates a view of a fastening component attached to the exterior of a second band of the incontinence garment.

### DETAILED DESCRIPTION OF THE PREFERRED EMBODIMENT

An incontinence garment that secures and conceals an absorbent pad, yet looks and fits like normal, stylish underwear.

An incontinence garment that can securely hold almost any brand, style or type of absorbent pad the wearer desires.

An incontinence garment that can be worn either with an absorbent pad for mild, moderate or sporadic incontinence, or without an absorbent pad as standard underwear when urinary incontinence protection is not needed.

An incontinence garment that has at least a first band and a second band attached below the waistband of a front panel and a rear panel. The first and second bands being made of an elastic type material and having a fastening component, such as a Velcro™ type hook and loop fastener. The hook portion of the fastening component is pre-attached to the first and second bands and the loop portion is attached to the absorbent pad by the wearer. The wearer then secures the absorbent pad by aligning the hook and loop portions of the fastening component.

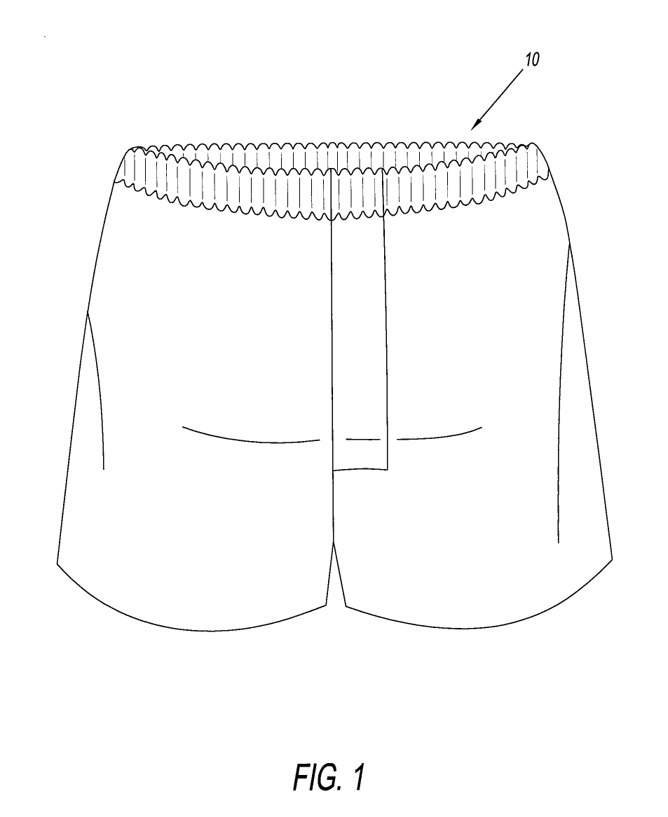

Referring to the drawings, an incontinence garment according to the present disclosure is shown and generally referenced to by numeral 10. FIG. 1 shows one embodiment of incontinence garment 10, a modified boxer short for men. However, incontinence garment 10 can be any style of men's undergarment desired, including but not limited to a smaller brief, a boxer-brief, as well as a short that can be used for running and sports. It is also contemplated that incontinence garment 10 is designed for women and available in multiple styles of women's undergarments (not shown).

Incontinence garment 10 is made to look and fit like a normal, standard pair of underwear while securing and concealing an absorbent pad used for mild to moderate urinary incontinence. Unlike other products available, incontinence garment 10 is also designed to be worn when UI protection is not needed. Thus, incontinence garment 10 can be conveniently worn as an everyday pair of underwear without an absorbent pad.

Incontinence garment 10 can hold nearly any brand or style of absorbent pad. In contrast, other products are specifically designed for a given manufacturers product, thus limiting the consumers choice and raising the cost to the consumer. Furthermore, incontinence garment 10 eliminates the need for a wearer to purchase multiple styles of underwear, and there is no need to change if an incontinence issue were to arise. For example, UI issues may occur unexpectedly or as a short-term problem due to surgery or a health condition. Incontinence garment 10 can replace the wearer's existing underwear wardrobe with a single style.

Incontinence garment 10 is preferably made from 100% pure cotton, although a wide range of natural or synthetic materials or fabrics can be used, such as cotton/polyester blends, nylons or any kind of silk, rayon or other soft light material. In order to reduce irritation and eliminate the feel of wearing an adult diaper, incontinence garment 10 does not contain any plastic. Furthermore, to be indistinguishable from traditional underwear, the material or fabric of incontinence garment 10 can be designed in a wide variety of stylish colors and patterns, such as, plaids, stripes and checks. Incontinence garment 10 is available in a range of sizes, including small, medium, large and extra-large.

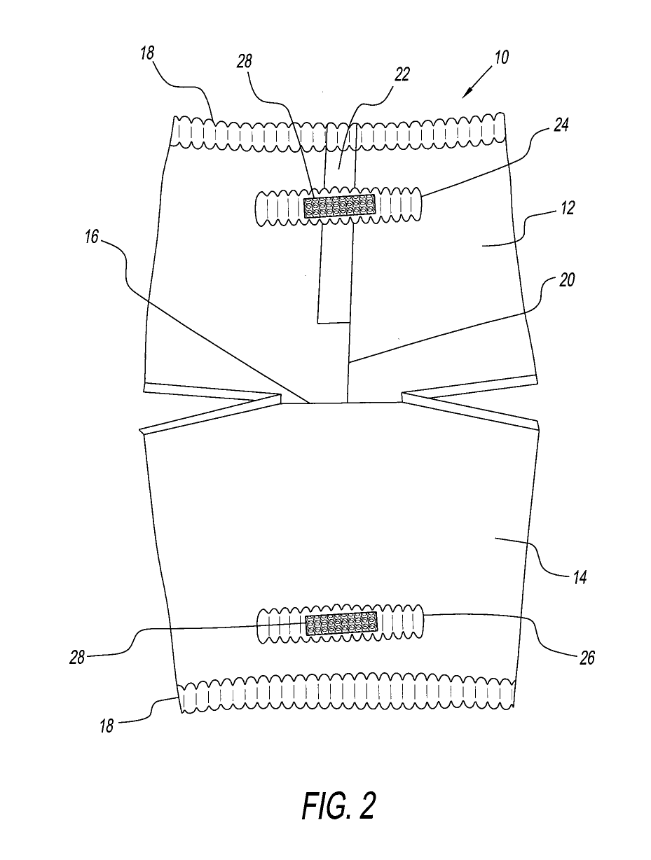

Referring to FIGS. 2, 4 and 5, incontinence garment 10 has a front section 12, a rear section 14 and a crotch area 16. Incontinence garment 10 has a waistband 18 at the torso opening that is made from an elastic material, a drawstring or a combination thereof. Front section 12 may contain a front seam 20 that runs from crotch area 16 to waistband 18. As shown in FIG. 2, front seam 20 may contain an opening or fly 22 on incontinence garment 10 that has been designed for men.

Furthermore, incontinence garment 10 has at least a first band 24 on the interior of front panel 12 and a second band 26 on the interior of rear panel 14. First band 24 and second band 26 are made of an elastic material and covered in a fabric for the comfort of the wearer. Preferably, first band 24 and second band 26 are covered in a fabric that matches the fabric used in the making of incontinence garment 10. First band 24 and second band 26 can be attached by any securing method, including but not limited to, sewing, gluing, heat seaming or with a fastener. Preferably, first band 24 and second band 26 are sewn onto front panel 12 and rear panel 14, respectively.

First band 24 and second band 26 are positioned about 2 inches to about 3 inches below waistband 18, depending on the style and size of incontinence garment 10. Preferably, first band 24 and second band 26 are about 2 inches below waistband 18. First band 24 and second band 26 are about 5 inches to about 6.5 inches in length and about 1 inch to about 3 inches high. Preferably, first band 24 and second band 26 are about 5 inches wide and 1.5 inches high. First band 24 and second band 26 are attached at the shorter ends to the fabric of front panel 12 and rear panel 14, respectively, as shown in FIG. 2.

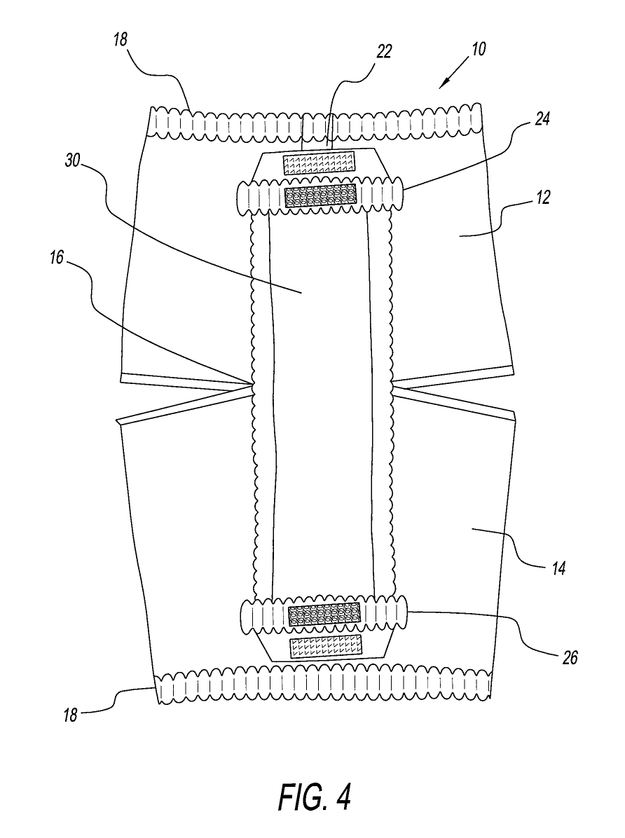

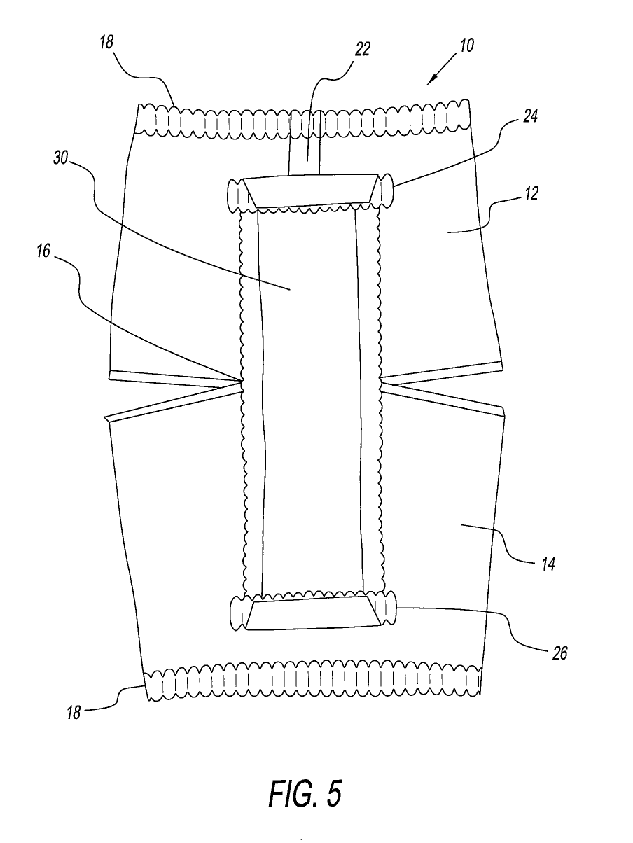

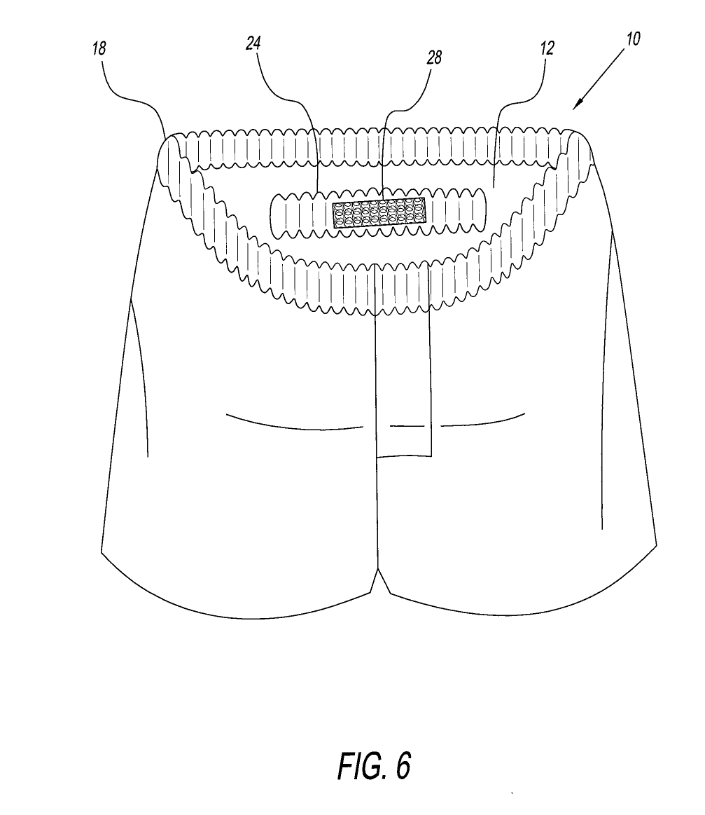

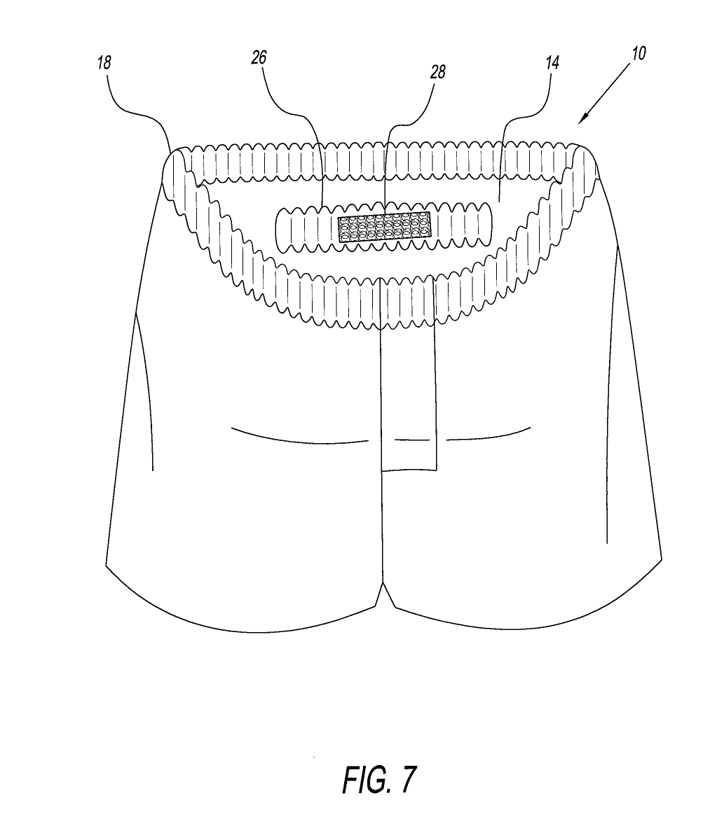

As a result of attaching first band 24 and second band 26 to the fabric, a loop type structure is formed within incontinence garment 10, as shown in FIGS. 6 and 7. The loop type structure measures about 4 inches to about 6 inches wide from first band 24 or second band 26 to the fabric of incontinence garment 10, and can increase in size as pressure is placed on the elastic material. The loop type structure holds an absorbent pad 30 within incontinence garment 10, as shown in FIGS. 4 and 5. The loop type structure and the preferred use of elastic material for first band 24 and second band 26 allows any style, size or shape of absorbent pad 30 to be used by the wearer. Absorbent pad 30 is secured into incontinence garment 10 by a fastening component 28. Fastening component 28 is preferably a Velcro type hook and loop fastener, but can also include other components used to fasten such as, adhesives, snaps, buttons and the like.

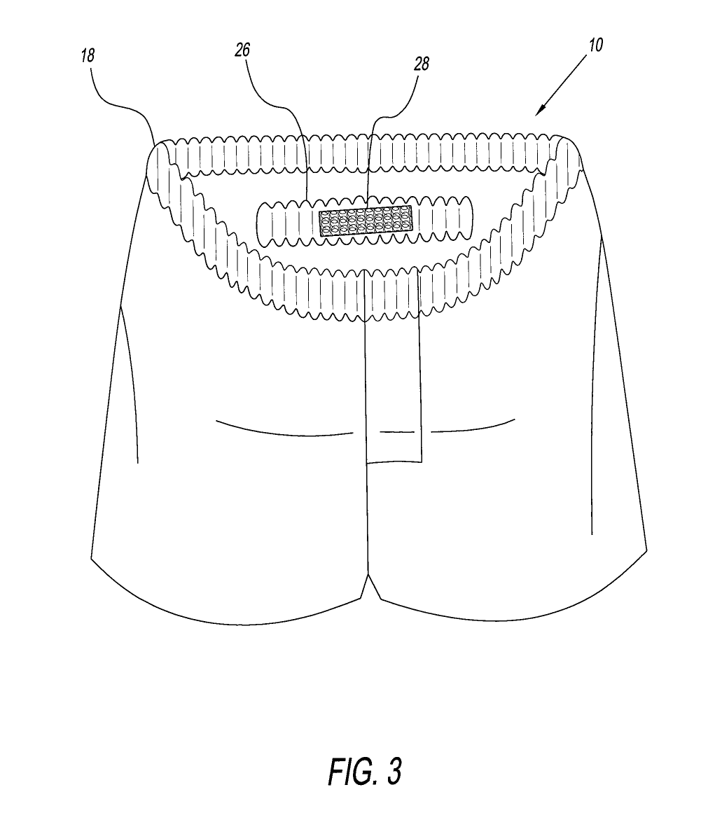

The hook portion of fastening component 28 is pre-attached to first band 24 and second band 26. Preferably, the hook portion of fastening component 28 is pre-attached to the interior surface of first band 24 and second band 26 that faces away from the wearer's body, as shown in FIG. 3. The loop portion of fastening component 28 is provided to the wearer as a precut piece that is then placed by the wearer on the two ends of absorbent pad 30. The loop portion of fastening component 28 has an adhesive back that becomes permanently attached to absorbent pad 30. Preferably, loop portion of fastening component 28 is attached to the absorbent surface of absorbent pad 30.

Absorbent pad 30 is secured within incontinence garment 10 by aligning the hook and loop portions of fastening component 28, as shown in FIG. 4. Because the wearer can attach the loop portion of fastening component 28 at any location on absorbent pad 30 there is a great amount of flexibility and room for adjustment when inserting and attaching absorbent pad 30 within incontinence garment 10. This, in turn, creates greater comfort and protection from leaks for the wearer during use. In addition, the use of a hook and loop type fastening component 28 provides the wearer with both a secure hold and ease when removing and replacing absorbent pad 30 as needed.

The user may also attach the loop portion of fastening component 28 to the ends of the non-absorbent surface of absorbent pad 30. Absorbent pad 30 is then placed in front of first band 24 and second band 26 with the loop portion of fastening component 28 being slightly above first band 24 and second band 26. The ends of absorbent pad 30 are pressed over the top of first band 24 and second band 26 and down into the loop type structure to align and secure the hook and loop portions of fastening component 28. The portion of absorbent pad 30 that goes over the top of first band 24 and second band 26 absorbs a portion of weight being placed on fastening 28 during use. This provides the wearer with a more secure hold and added comfort that the pad will remain in place.

In another embodiment, the hook portion of fastening component 28 is pre-attached to the exterior surface of first band 24 and second band 26 that faces the wearer's body, as shown in FIGS. 6 and 7. The loop portion of fastening component 28 is placed on the absorbent surface of absorbent pad 30. Absorbent pad 30 is secured to incontinence garment 10 by being placed under first band 24 and second band 26 and then wrapping the ends around the bands to align the hook and loop portions of fastening component 28, as show in FIG. 5.

The dimensions and give of the elastic material of first band 24 and second band 26, along with the use of fastening component 28, allows nearly any brand, style and weight of absorbent pad 30 to fit within incontinence garment 10. The wearer is not limited to a product-specific absorbent pad 30 and can select the one they prefer. As a result, the wearer will likely save money and obtain a more comfortable fit.

Furthermore, the use of the elastic material of first band 24 and second band 26 is designed to provide additional security. As absorbent pad 30 captures liquid, its weight will gradually increase. The give of the elastic material of first band 24 and second band 26 provides relief from the strain that would be placed on fastening component 28. Therefore, it is far less likely that fastening component will release during use. This extra secure hold provides the wearer with added confidence that absorbent pad 30 will remain in place.

The present disclosure having been thus described with particular reference to the preferred forms thereof, it will be obvious that various changes and modifications may be made therein without departing from the spirit and scope of the present disclosure as defined in the disclosure.

What is claimed is:

1. An incontinence garment comprising:
a waistband;
a front portion attached to said waistband, wherein said front portion has at least a first securing member disposed thereon;
a back portion attached to said waistband in a position opposite to said front portion, wherein said back portion has at least a second securing member disposed thereon;
a crotch portion disposed between said front portion and said back portion and opposite to said waistband, wherein said first and second securing members are disposed between said waistband and said crotch portion; and
an absorbent assembly disposed between said front portion and second back portion, wherein said absorbent assembly comprises a first end affixed to said first securing member and a second end affixed to said second securing member.

2. The garment according to claim 1, wherein said first and second securing members are formed of an elastic type material.

3. The garment according to claim 1, where said first end of said absorbent assembly is affixed to said first securing member via a first fastener, and said second end of said absorbent assembly is affixed to said second securing member via a second fastener.

4. The garment according to claim 1, wherein said first and second fasteners are at least one fastener selected from the group consisting of: hook and loop type fasteners, adhesives, snaps, and buttons.

5. The garment according to claim 4, wherein said first and second fasteners are hook and loop type fasteners, wherein each said first and second securing members have respective hook portions from said hook and loop type fastener affixed thereto and each said first and second ends of said absorbent assembly have respective loop portions affixed thereto.

6. The garment according to claim 1, wherein said absorbent assembly includes an absorbent pad.

7. The garment according to claim 5, wherein said hook portions are affixed to a surface of said first and second securing members, respectively, wherein said surface is that surface which faces toward the user's body.
